(19) United States
(12) Patent Application Publication
(10) Pub. No.: US 2019/0388033 A1
(43) Pub. Date: Dec. 26, 2019

(54) OPTICAL FORCE SENSING CATHETER SYSTEM

(71) Applicant: ST. JUDE MEDICAL INTERNATIONAL HOLDING S.Á R.L., Luxembourg (LU)

(72) Inventor: Xiangyang Zhang, Maple Grove, MN (US)

(21) Appl. No.: 16/481,673

(22) PCT Filed: Feb. 2, 2018
(86) PCT No.: PCT/IB2018/050682

§ 371 Date: Jul. 29, 2019

Related U.S. Application Data
(60) Provisional application No. 62/454,396, filed on Feb. 3, 2017.

(51) Int. Cl. A61B 5/00; A61M 25/00; A61B 18/14

(52) U.S. Cl. CPC A61B 5/6852; A61B 5/6885; A61B 2090/065; A61B 18/1492; A61M 2025/0002; A61M 25/0074

(57) ABSTRACT

Aspects of the present disclosure are directed toward systems and methods for calibrating and detecting force applied to a distal tip of a medical catheter. Some embodiments are directed toward a medical catheter with a deformable body near a distal tip of the catheter that deforms in response to a force applied at the distal tip. A force sensor detects various components of the deformation and processor circuitry, based on the detected components of the deformation, determines a force applied to the distal tip of the catheter and accounts for the effects of a bending moment.

### CROSS-REFERENCE TO RELATED APPLICATIONS

This application is a continuation of International Application No. PCT/IB2018/050682, filed 2 Feb. 2018, which claims the benefit of U.S. provisional application No. 62/454,396, filed 3 Feb. 2017.

### BACKGROUND

a. Field

The instant disclosure relates generally to force sensing systems capable of determining a force applied at a distal tip of a medical catheter. More specifically, the invention relates to algorithms for determining a force exerted on a catheter tip based on a number of deformation measurements.

b. Background Art

Exploration and treatment of various organs or vessels is possible using catheter-based diagnostic and treatment systems. Such catheters are introduced through a vessel leading to the cavity of the organ to be explored or treated, or alternatively may be introduced directly through an incision made in the wall of the organ. In this manner, the patient avoids the trauma and extended recuperation times typically associated with open surgical procedures.

To provide effective diagnosis or therapy, it is frequently necessary to first map the zone to be treated with great precision. Such mapping may be performed, for example, when it is desired to selectively ablate current pathways within a heart to treat atrial fibrillation. Often, the mapping procedure is complicated by difficulties in locating the zone(s) to be treated due to periodic movement of the heart throughout the cardiac cycle.

Mapping systems often rely on manual feedback of the catheter and/or impedance measurements to determine when the catheter is properly positioned in a vessel or organ. These systems do not measure contact forces with the vessel or organ wall, or detect contact forces applied by the catheter against the organ or vessel wall that may modify the true wall location. Accordingly, the mapping may be in accurate due to artifacts created by excessive contact force.

To facilitate improved mapping, it is desirable to detect and monitor contact forces between a catheter tip and a wall of an organ or vessel to permit faster and more accurate mapping. Once the topography of the vessel or organ is mapped, either the same or a different catheter may be employed to effect treatment. Depending upon the specific treatment to be applied to the vessel or organ, the catheter may comprise any of a number of end effectors, such as, for example, RF ablation electrodes, mapping electrodes, etc.

The effectiveness of such end effectors often depends on having the end effector in contact with the tissue of the wall of the organ or vessel, which is inherently unstable due to the motion of the organ or vessel. Existing catheter-based force sensing systems often don't have the ability to accurately sense the load applied to the distal tip of the catheter associated with either movement of the catheter or the tissue wall in contact therewith.

For example, in the case of a cardiac ablation system, the creation of a gap between the end effector of the treatment system and the tissue wall may render the treatment ineffective, and inadequately ablate the tissue zone. Alternatively, if the end effector of the catheter contacts the tissue wall with excessive force, it may inadvertently puncture the tissue.

In view of the foregoing, it would be desirable to provide a catheter-based diagnostic or treatment system that permits sensing of the load applied to the distal extremity of the catheter, including periodic loads arising from movement of the organ or tissue. It is further desirable to provide diagnostic and treatment apparatus that permit computation of forces applied to a distal tip of a catheter with reduced sensitivity to the location on the catheter tip at which the forces are applied.

The foregoing discussion is intended only to illustrate the present field and should not be taken as a disavowal of claim scope.

### BRIEF SUMMARY

Aspects of the present disclosure are directed toward systems and methods for calibrating and detecting force applied to a distal tip of a medical catheter using a fiber-optic force sensor and processor circuitry. In particular, the instant disclosure relates to a deformable body near a distal tip of a medical catheter that deforms in response to a force applied at the distal tip. The fiber-optic force sensor detects various components of the deformation and the processor circuitry, based on the detected components of the deformation, determines a force applied to the distal tip of the catheter.

Various embodiments of the present disclosure are directed to force-sensing catheter systems. One such system includes a catheter with a distal tip, a deformable body coupled near the distal tip, a force sensor with three or more sensing elements, and processing circuitry. The deformable body deforms in response to a force exerted on the distal tip. The force sensor detects the deformation of the deformable body in response to the force exerted at various locations of the deformable body, and transmits a signal indicative of the deformation. The processing circuitry receives the signal from each of the force sensing elements, indicative of the deformation, and determines a magnitude of the force exerted on the catheter tip. The processing circuitry further accounts for a bending moment, associated with the exerted force, exerted upon the deformable body. In more specific embodiments, the force-sensing catheter system further includes a display communicatively coupled to the processing circuitry, that visually indicates to a clinician the force exerted on the distal tip of the catheter.

Some embodiments of the present disclosure are directed to calibration methods for a force-sensing catheter system. One such method includes successively applying forces at designated points along a distal tip of a catheter, and based on a response of a force sensor to the force applications, determine a first compliance matrix. In one specific/experimental embodiment, the calibration method further includes determining a second compliance matrix associated with a moment, ( [symbol] ), based on the force sensors response to the force applications. In the present embodiment, the force sensor includes three sensing elements, and the first compliance matrix is associated with a force, ( [symbol] ).

Yet other embodiments disclosed herein are directed to methods for detecting a force and a moment exerted on a distal tip of a force-sensing catheter system. In one embodiment, the method for detecting a force and moment exerted on a distal tip of a force-sensing catheter system includes receiving three or more signals indicative of the displacement measured on the distal tip of the force-sensing catheter, and applying a compliance matrix to the measured displacement to determine the force and moment exerted on the distal tip. In a more detailed embodiment, the step of receiving three or more signals indicative of the displacement measured on the distal tip of the force-sensing catheter includes receiving five signals, and the step of applying a compliance matrix to the measured displacements to determine the force and moment exerted on the distal tip utilizes the equation:

$$\{ \begin{matrix}{F}_{x} \\ {F}_{y} \\ {F}_{z} \\ {M}_{x} \\ {M}_{y}\end{matrix} \} = {\{ \begin{matrix}{C}_{11} & {C}_{12} & {C}_{13} & {C}_{14} & {C}_{15} \\ {C}_{21} & {C}_{22} & {C}_{23} & {C}_{24} & {C}_{25} \\ {C}_{31} & {C}_{32} & {C}_{33} & {C}_{34} & {C}_{35} \\ {C}_{41} & {C}_{42} & {C}_{43} & {C}_{44} & {C}_{45} \\ {C}_{51} & {C}_{52} & {C}_{53} & {C}_{54} & {C}_{55}\end{matrix} \}}^{- 1} \cdot \{ \begin{matrix}{d}_{1} \\ {d}_{2} \\ {d}_{3} \\ {d}_{4} \\ {d}_{5}\end{matrix} \}$$

where the \tilde{C} matrix is the compliance matrix.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a diagrammatic overview of a system for force sensing, consistent with various embodiments of the present disclosure;

FIG. 2 is a block diagram of a force sensing system, consistent with various embodiments of the present disclosure;

FIG. 2A is a schematic depiction of an interferometric fiber optic sensor, consistent with various embodiments of the present disclosure;

FIG. 2B is a schematic depiction of a fiber Bragg grating optical strain sensor, consistent with various embodiments of the present disclosure;

FIG. 3 is a partial cutaway view of a distal portion of a catheter assembly having a fiber optic force sensing assembly, consistent with various embodiments of the present disclosure;

FIG. 4 is a front view of a fiber optic force sensing assembly, consistent with various embodiments of the present disclosure;

FIG. 4A is a top view of the fiber optic force sensing assembly of FIG. 4, consistent with various embodiments of the present disclosure;

FIG. 4B is a cross-sectional side-view of a Fabry-Perot strain sensor within the fiber optic force sensing assembly of FIG. 4, consistent with various embodiments of the present disclosure; and

FIG. 5 is an isometric side view of a catheter tip assembly, consistent with various embodiments of the present disclosure.

While various embodiments discussed herein are amenable to modifications and alternative forms, aspects thereof have been shown by way of example in the drawings and will be described in further detail. It should be understood, however, that the intention is not to limit the disclosure to the particular embodiments described. On the contrary, the intention is to cover all modifications, equivalents, and alternatives falling within the scope of the disclosure including aspects defined in the claims.

### DETAILED DESCRIPTION OF EMBODIMENTS

Aspects of the present disclosure are directed toward systems and methods for calibrating and detecting force applied to a distal tip of a medical catheter. In particular, the instant disclosure relates to a deformable body near a distal tip of a medical catheter that deforms in response to a force applied at the distal tip. Force sensors, such as fiber-optic force sensors, detect various components of the deformation, and processor circuitry, based on the detected components of the deformation, determines a force applied to the distal tip of the catheter. Importantly, various aspects of the present disclosure are directed to accounting for the effect of a bending moment on the force sensor. Details of the various embodiments of the present disclosure are described below with specific reference to the figures.

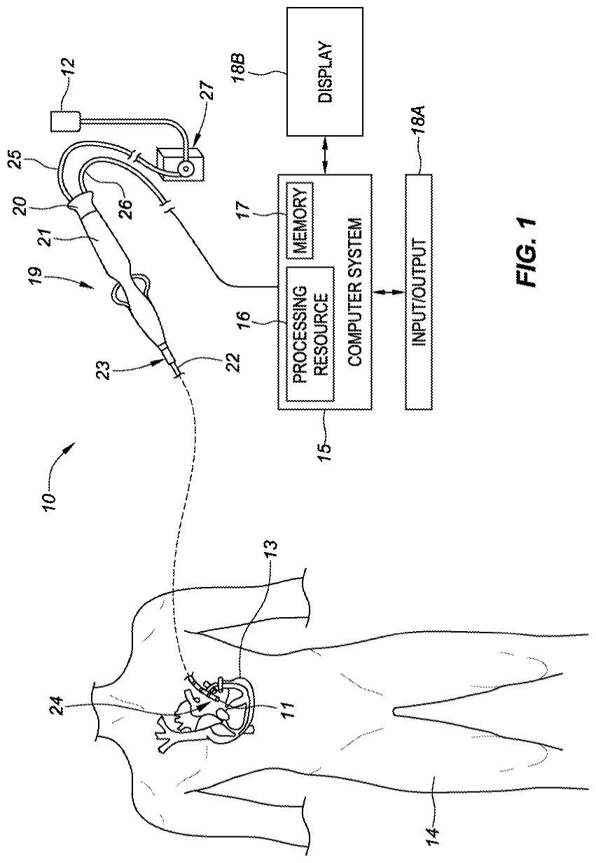

Referring now to the drawings wherein like reference numerals are used to identify identical components in the various views, FIG. 1 generally illustrates a system 10 for force detection. The system 10 includes an elongate medical device 19 with a fiber optic force sensor assembly 11 configured to be used in the body for medical procedures. The fiber optic force sensor assembly 11 is included as part of a medical device, such as an elongate medical device 19, and may be used for diagnosis, visualization, and/or treatment of tissue 13 (such as cardiac or other tissue) in the body. For example, the medical device 19 may be used for ablation therapy of tissue 13 or mapping purposes in a patient's body 14. FIG. 1 further shows various sub-systems included in the overall system 10. The system 10 may include a main computer system 15 (including an electronic control unit 16 (e.g., processing resource) and data storage 17 (e.g., memory)). The computer system 15 may further include conventional interface components, such as various user input/output mechanisms 18A and a display 18B, among other components. Information provided by the fiber optic force sensor assembly 11 may be processed by the computer system 15 and may provide data to the clinician via the input/output mechanisms 18A and/or the display 18B, or in other ways as described herein. Specifically, the display 18B may visually communicate a force exerted on the elongated medical device 19—where the force exerted on the elongated medical device 19 is detected in the form of a deformation of at least a portion of the elongated medical device by the fiber optic force sensor assembly 11, and the measured deformations are processed by the computer system 15 to determine the force exerted.

In the illustrative embodiment of FIG. 1, the elongated medical device 19 may include a cable connector or interface 20, a handle 21, a tubular body or shaft 22 having a proximal end 23 and a distal end 24. The elongated medical device 19 may also include other conventional components not illustrated herein, such as a temperature sensor, additional electrodes, and corresponding conductors or leads. The connector 20 may provide mechanical, fluid and/or electrical connections for cables 25, 26 extending from a fluid reservoir 12 and a pump 27 and the computer system 15, respectively. The connector 20 may comprise conventional components known in the art and, as shown, may be disposed at the proximal end of the elongate medical device 19.

The handle 21 provides a portion for a user to grasp or hold elongated medical device 19 and may further provide a mechanism for steering or guiding the shaft 22 within the patient's body 14. For example, the handle 21 may include a mechanism configured to change the tension on a pull-wire extending through the elongate medical device 19 to the distal end 24 of the shaft 22 or some other mechanism to steer the shaft 22. The handle 21 may be conventional in the art, and it will be understood that the configuration of the handle 21 may vary. In one embodiment, the handle 21 may be configured to provide visual, auditory, tactile and/or other feedback to a user based on information received from the fiber optic force sensor assembly 11. For example, if contact to tissue 13 is made by distal tip 24, the fiber optic force sensor assembly 11 will transmit data to the computer system 15 indicative of the contact. In response to the computer system 15 determining that the data received from the fiber optic force sensor assembly 11 is indicative of contact between the distal tip 24 and a patient's body 14, the computer system 15 may operate a light-emitting-diode on the handle 21, a tone generator, a vibrating mechanical transducer, and/or other indicator(s), the outputs of which could vary in proportion to the amount of force sensed at the electrode assembly.

The computer system 15 may utilize software, hardware, firmware, and/or logic to perform a number of functions described herein. The computer system 15 can be a combination of hardware and instructions (e.g., software) to share information. The hardware, for example can include processing resource 16 and/or a memory 17 (e.g., non-transitory computer-readable medium (CRM) database, etc.). A processing resource 16, as used herein, may include a number of processors capable of executing instructions stored by the memory resource 17. Processing resource 16 may be integrated in a single device or distributed across multiple devices. The instructions (e.g., computer-readable instructions (CRI)) can include instructions stored on the memory 17 and executable by the processing resource 16 for force detection.

The memory resource 17 can be in communication with the processing resource 16. A memory 17, as used herein, can include a number of memory components capable of storing instructions that can be executed by processing resource 16. Such a memory 17 can be a non-transitory computer readable storage medium, for example. The memory 17 can be integrated in a single device or distributed across multiple devices. Further, the memory 17 can be fully or partially integrated in the same device as the processing resource 16 or it can be separate but accessible to that device and the processing resource 16. Thus, it is noted that the computer system 15 can be implemented on a user device and/or a collection of user devices, on a mobile device and/or a collection of mobile devices, and/or on a combination of user devices and mobile devices.

The memory 17 can be in communication with the processing resource 16 via a communication link (e.g., path). The communication link can be local or remote to a computing device associated with the processing resource 16. Examples of a local communication link can include an electronic bus internal to a computing device where the memory 17 is one of a volatile, non-volatile, fixed, and/or removable storage medium in communication with the processing resource 16 via the electronic bus.

In various embodiments of the present disclosure, the computer system 15 may receive optical signals from a fiber optic force sensor assembly 11 via one or more optical fibers extending a length of the catheter shaft 22. A processing resource 16 of the computer system 15 will execute an algorithm stored in memory 17 to compute a force exerted on catheter tip 24 that is devoid of error associated with a bending moment exerted on the fiber optic force sensor assembly 11, based on the received optical signals.

U.S. Pat. No. 8,567,265 discloses various optical force sensors for use in medical catheter applications, such optical force sensors are hereby incorporated by reference as though fully disclosed herein. These optical force sensors may be used in accordance with the algorithms disclosed herein to detect a force exerted on a catheter tip and to filter out error in the measured force associated with the placement of the force on the catheter tip relative to the fiber optic force sensor assembly 11.

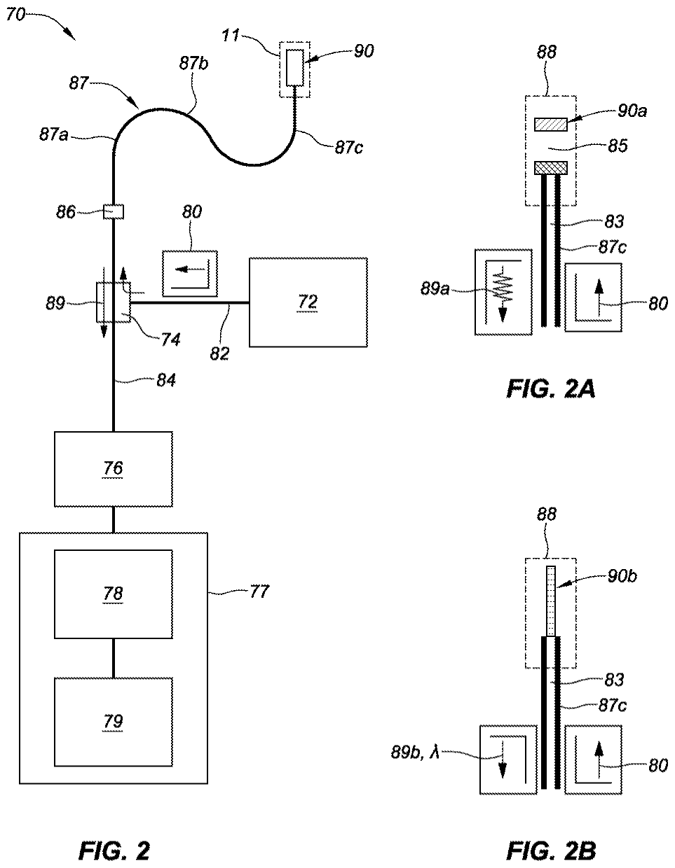

Referring to FIG. 2, an embodiment of a force sensing system 70 is depicted in accordance with the invention. The force sensing system 70 may comprise an electromagnetic source 72, a coupler 74, a receiver 76, an operator console 77 operatively coupled with a microprocessor 78 and a storage device 79. The electromagnetic source 72 outputs a transmitted radiation 80 of electromagnetic radiation that is substantially steady state in nature, such as a laser or a broadband light source. A transmission line 82 such as a fiber optic cable carries the transmitted radiation 80 to the coupler 74, which directs the transmitted radiation 80 through a transmitting/receiving line 84 and through a fiber optic element 83 (see, e.g., FIG. 2A) contained within a flexible, elongate catheter assembly 87 to a fiber optic force sensing element 90 within a fiber optic force sensor assembly 11. It is to be understood that while various embodiments of the present disclosure are directed to force sensing systems with fiber optic force sensing elements for detecting a change in dimension (e.g., deformation) of a catheter assembly 87, various other embodiments may include non-fiber optic based measurement systems as are well known in the art. Moreover, it is to be understood that the force sensing elements (also referred to as sensing elements) measure the deformation of a deformable body (e.g., a distance or displacement), and do not directly measure a force. The catheter assembly 87 may include one or more transmitting/receiving lines 84 coupled to one or more fiber optic elements 83 within the fiber optic force sensor assembly 11. The fiber optic element(s) 83 of the catheter assembly 87 and transmitting/receiving(s) line 84 may be coupled through a connector 86 as depicted in FIG. 2.

The catheter assembly 87 may have a width and a length suitable for insertion into a bodily vessel or organ. In one embodiment, the catheter assembly 87 comprises a proximal portion 87 a, a middle portion 87 b and a distal portion 87 c. The distal portion 87 c may include an end effector which may house the fiber optic force sensor assembly 11 and the one or more fiber optic force sensing element(s) 90. The catheter assembly may be of a hollow construction (i.e. having a lumen) or of a non-hollow construction (i.e. no lumen), depending on the application.

In response to a deformation of a deformable body, due to a force being exerted on a distal tip of a catheter, one or more fiber optic elements 90 within the fiber optic force sensor assembly 11 will modulate the radiation received from the transmission line 82 and transmit the modulated radiation to the operator console 77 via receiving lines 84. Once the radiation is received by the operator console 77, a microprocessor 78 may run an algorithm stored on storage device 79 to detect a force exerted on the catheter tip, and to determine and remove an error associated with a bending moment placed on the fiber optic force sensor assembly 11 from the determined force exerted on the catheter tip.

A fiber optic force sensing element 90 for detecting a deformation of a deformable body may be an interferometric fiber optic strain sensor, a fiber Bragg grating strain sensor, or other fiber optic sensor well known in the art.

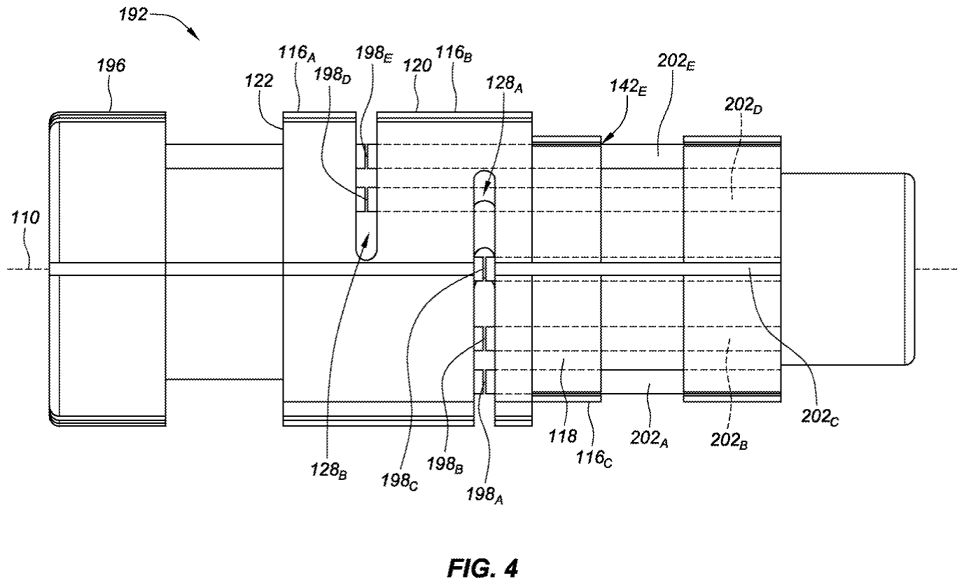

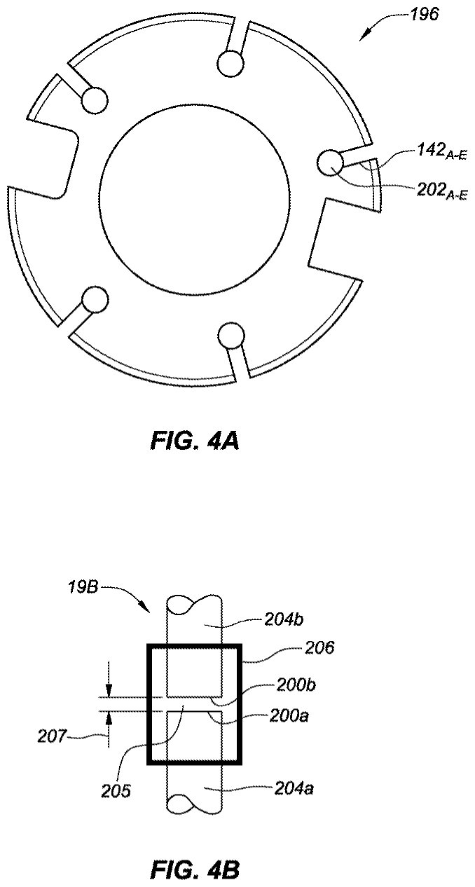

Referring to FIG. 2A, fiber optic force sensing assembly 88 includes an interferometric fiber optic strain sensor 90a. In this embodiment, the transmitted radiation 80 enters an interferometric gap 85 within the interferometric fiber optic strain sensor 90a. A portion of the radiation that enters the interferometric gap 85 is returned to the fiber optic cable of the catheter assembly 87c as a modulated waveform 89a. The various components of the interferometric fiber optic strain sensor 90 may comprise a structure that is integral to fiber optic element 83 (see, e.g., FIG. 4B). Alternatively, the fiber optic element 83 may cooperate with the structure to which it is mounted to form the interferometric gap 85.

Referring to FIG. 2B, fiber optic force sensing assembly 88 includes a fiber Bragg grating strain sensor 88. In this embodiment, the transmitted radiation 80 enters a fiber Bragg grating 90b, the gratings of which are typically integral with the fiber optic element 83 and reflect only a portion 89b of the transmitted radiation 80 about a central wavelength λ. The central wavelength λ at which the portion 89b is reflected is a function of the spacing between the gratings of the fiber Bragg grating. Therefore, the central wavelength λ is indicative of the strain on the fiber Bragg grating strain sensor 88 relative to some reference state.

The reflected radiation 89, be it the modulated waveform 89a (as in FIG. 2A) or the reflected portion 89b (as in FIG. 2B), is transmitted back through the transmitting/receiving line 84 to the receiver 76 (as shown in FIG. 2). The strain sensing system 70 may interrogate the one or more fiber optic strain sensing element(s) 90 at an exemplary and non-limiting rate of 10-Hz. The receiver 76 is selected to correspond with the type of strain sensing element 90 utilized. That is, the receiver may be selected to either detect the frequency of the modulated waveform 89a for use with the interferometric fiber optic strain sensor of FIG. 2A, or to resolve the central wavelength of the reflected portion 89b for use with fiber Bragg grating strain sensor of FIG. 2B. The receiver 76 manipulates and/or converts the incoming reflected radiation 89 into digital signals for processing by microprocessor 78.

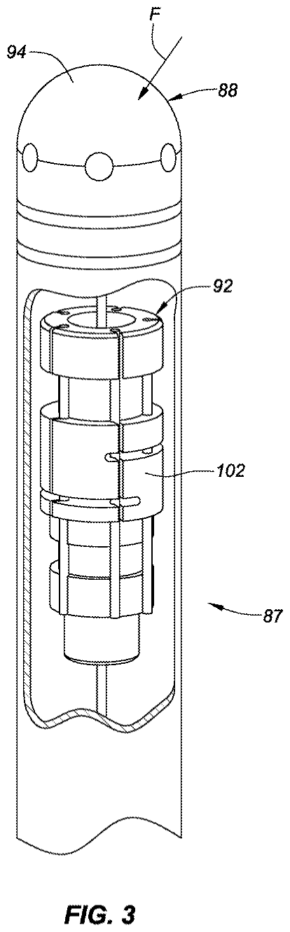

Referring to FIG. 3, an example of an end effector 88 including an ablation head 88 and a fiber optic force sensor assembly 92 is depicted. The fiber optic force sensor assembly 92 may be integral with a structural member 102, also referred to as a deformable body, that deforms measuredly in response to a force F imposed on a distal extremity 94 of the catheter (e.g., when distal extremity 94 contacts the wall of a bodily vessel or organ).

It is understood that one or more end effectors 88 of different kinds, e.g., mapping electrodes or ablation electrodes, such as are known in the art for diagnosis or treatment of a vessel or organ may be utilized with the present invention. For example, the catheter assembly 87 may be configured as an electrophysiology catheter for performing cardiac mapping and ablation. In other embodiments, the catheter assembly 87 may be configured to deliver drugs or bioactive agents to a vessel or organ wall or to perform minimally invasive procedures such as, for example, cryo-ablation.

Referring to FIGS. 4 and 4A, the fiber optic force sensing assembly 192 includes structural member 196 and a plurality of fiber optics 202_{A-E}. In this embodiment, the structural member 196 defines a longitudinal axis 110. The structural member 196 is divided into a plurality of segments 116_{A-C}, a distal segment, a proximal segment, and a base segment, respectively. The segments 116_{A-C} may be adjacent each other in a serial arrangement along the longitudinal axis 110.

The segments 116_{A-C} may be bridged by a plurality of flexure portions 128, identified individually as flexure portions 128_{A-B}, thus defining a plurality of neutral axes. Each neutral axis constitutes the location within the respective flexure portions 128_{A-B} that the stress is zero when subject to pure bending in any direction.

In one embodiment, adjacent members of the segments 116_{A-C} may define a plurality of gaps at the flexure portions 128_{A-B}, each having a separation dimension. It is noted that while the separation dimensions of the gaps are depicted as being uniform, the separation dimensions may vary in the lateral direction across a given gap. A central plane is located equidistant between adjacent ones of the segments 116_{A-C}.

Structural member 196 may include a plurality of grooves 142_{A-E} that are formed within the outer surface of the structural member. The grooves 142_{A-E} may be spaced rotationally equidistant (i.e. spaced 72° apart where there are five grooves) about longitudinal axis 110, and may be oriented in a substantially axial direction along the structural member 196. Each of the grooves may terminate at a respective one of the gaps of the flexure portions 128_{A-B}. For example, grooves 142_{D-E} may extend along the base segment 118 and the proximal segment 120 terminating at the gap at flexure portion 128_{B}. Other grooves, such as grooves 142_{A-C}, may extend along the base segment 118 and terminate at the gap at flexure portion 128_{A}.

The fiber optics 202_{A-E} define a plurality of light propagation axes and distal ends. The fiber optics 202_{A-E} may be disposed in the grooves 142_{A-E}, respectively, such that the distal ends terminate at the gap of either flexure portion 128_{A-B}. For example, the fiber optic 202_{A} may extend along the groove 142_{A}, terminating proximate or within the gap at flexure portion 128_{A}. Likewise, fiber optic 202_{E} may extend along the groove 142_{E} and terminate proximate or within the gap at flexure portion 128_{B}. Surfaces of the flexure portions 128_{A-B} opposite the distal ends of the fiber optics 202_{A-E} may be made highly reflective.

The gaps at the flexure portion 128_{A-B} may be formed so that they extend laterally through a major portion of the structural member 196. Also, the gaps may be oriented to extend substantially normal to longitudinal axis 110 (as depicted) or at an acute angle with respect to the longitudinal axis. In the depicted embodiment, the structural member comprises a hollow cylindrical tube with the gaps comprising slots that are formed from one side of the hollow cylindrical tube and are transverse to the longitudinal axis 110, extending through the longitudinal axis 110 and across a portion of the inner diameter of the hollow cylindrical tube.

In FIG. 4, flexure portions 128 define a semi-circular segment. The depth of the flexure portions traverse the inner diameter of the hollow cylindrical tube and may be varied to establish a desired flexibility of the flexure. That is, the greater the depth of the flexure portions 128 the more flexible the flexure portions are. The flexure portions may be formed by one or more of the various ways available to a skilled artisan, such as but not limited to sawing, laser cutting or electro-discharge machining (EDM). The slots which form the flexure portions 128_{A-B} may be formed to define non-coincident neutral axes.

Referring to FIGS. 2, 4, and 4B, a fiber optic force sensor assembly 11 integral with a structural member 196 is depicted in an embodiment of the present disclosure. In some embodiments, the fiber optic force sensor assembly 11 includes fiber optics 202_{A-E}, each operatively coupled to a respective one of a plurality of Fabry-Perot strain sensors 19B, as shown in FIG. 4B.

The operation of a Fabry-Perot strain sensors 19B is depicted in FIG. 4B. The fiber optic is split into a transmitting element 204a and a reflecting element 204b, each being anchored at opposing ends of a hollow tube 206. The transmitting and reflecting elements 204a and 204b are positioned to define an interferometric gap 205 therebetween having an operative length 207. The free end of the transmitting element 204a may be faced with a semi-reflecting surface 200a, and the free end of the reflecting element 204b may be faced with a reflecting surface 200b.

The fiber optics may be positioned along the grooves 142_{A-E} (as shown in FIG. 4) so that the respective Fabry-Perot strain sensor 19B is bridged across one of the flexure portions 128_{A-B}. For example, fiber optic 202_{A} may be positioned within groove 142_{A} so that the Fabry-Perot strain sensor 19B bridges the gap at the flexure portion 198_{A} between a proximal segment 116_{E} and a base segment 116_{C}.

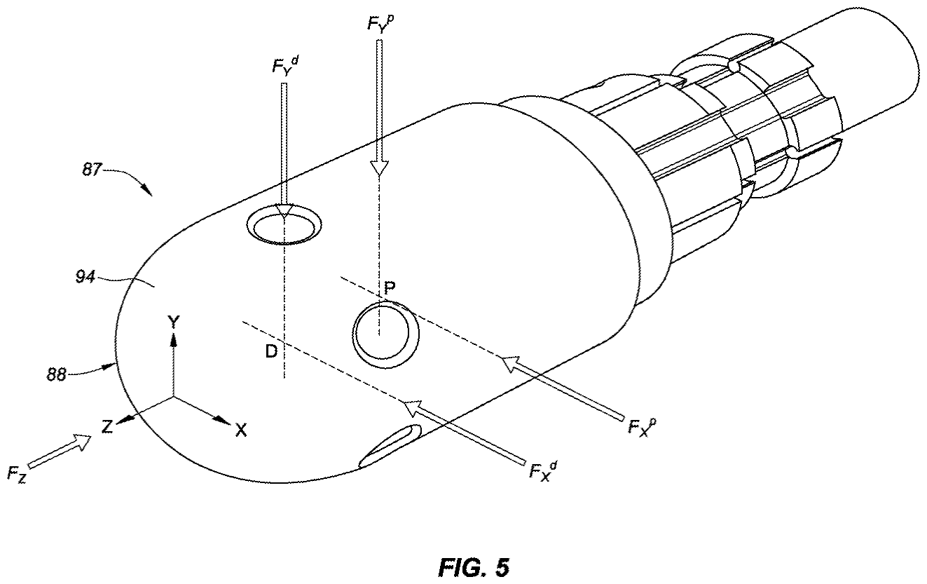

FIG. 5 is an isometric side view of a catheter tip assembly 87, consistent with various embodiments of the present disclosure. An end effector 88 comprises an ablation head for conducting tissue ablation within a patient's vasculature. To facilitate calibration of a fiber optic force sensor assembly within the catheter tip assembly, the catheter tip assembly 87 must be calibrated by loading the end effector 88 at five locations (e.g., where the force sensor includes five fiber optic force sensing elements 90). Specifically, and as discussed in more detail below, an axial load (F_{Z}) must be applied, and four lateral loads. Two lateral loads are applied at a distal plane D, and two lateral loads are applied at a proximal plane P. The lateral loads applied to each plane are applied at transverse angles relative to one another (e.g., 90 degrees). As discussed in more detail below, these initial measurements may be used to calibrate the force sensing system and to facilitate accurate force measurement of the end effector 88 in vivo. The calibration process corrects for the effect of a bending moment as applied to the force sensor assembly when a force is exerted on the end effector 88. Such a bending moment may negatively impact the accuracy of the resulting force calculation from the force-sensing system. The following algorithms address such bending moments by calibrating a force sensor assembly and/or accounting for such bending moments in force calculations based on the signals received from force sensing elements of a force sensor assembly.

### A Three Point Force Measurement System

In a force sensor assembly with three fiber optic force sensing elements, the measured displacement is correlated with applied force by:

D=\tilde{C}·F Equation 1

where D is the displacement vector, F is the force vector and \tilde{C} is the compliance tensor (matrix).

After calibration, the force can be calculated by:

F=\tilde{C}⁻¹·D [symbol] \tilde{K}·D Equation 2

where \tilde{K}=\tilde{C}⁻¹ is called the stiffness matrix and is obtained during calibration.

In an expanded format, Eq. (1) and (2) can be written as:

$$\begin{matrix}\{ \begin{matrix}{d}_{x} \\ {d}_{y} \\ {d}_{z}\end{matrix} \} = \{ \begin{matrix}{C}_{11} & {C}_{12} & {C}_{13} \\ {C}_{21} & {C}_{22} & {C}_{23} \\ {C}_{31} & {C}_{32} & {C}_{33}\end{matrix} \} \cdot \{ \begin{matrix}{F}_{x} \\ {F}_{y} \\ {F}_{z}\end{matrix} \} \\ \text{and:} & Equation 3 \\ \{ \begin{matrix}{F}_{x} \\ {F}_{y} \\ {F}_{z}\end{matrix} \} = \{ \begin{matrix}{K}_{11} & {K}_{12} & {K}_{13} \\ {K}_{21} & {K}_{22} & {K}_{23} \\ {K}_{31} & {K}_{32} & {K}_{33}\end{matrix} \} \cdot \{ \begin{matrix}{d}_{x} \\ {d}_{y} \\ {d}_{z}\end{matrix} \} & Equation 4\end{matrix}$$

The coordinate system embedded in a force sensor assembly with three fiber optic force sensing elements, such as the TactiCath™ contact force ablation catheter, sold by St. Jude Medical, Inc., includes an axial direction which is the z axis, and the x and y axes are two lateral directions with x in horizontal and y in vertical directions.

In a force sensor with three fiber optic force sensing elements, a calibration may be conducted with 3 known forces successively being exerted on a distal tip of the catheter. Under a certain force with a specific direction, the displacements are:

$$\{ \begin{matrix}{d}_{1 x} \\ {d}_{1 y} \\ {d}_{1 z}\end{matrix} \} = \{ \begin{matrix}{C}_{11} & {C}_{12} & {C}_{13} \\ {C}_{21} & {C}_{22} & {C}_{23} \\ {C}_{31} & {C}_{32} & {C}_{33}\end{matrix} \} \cdot \{ \begin{matrix}{F}_{1 x} \\ {F}_{1 y} \\ {F}_{1 z}\end{matrix} \} \qquad Equation 5$$

Repeating this with the other two directions and the relation between displacements and forces under 3 independent directions (not in the same plane) is:

$$\{ \begin{matrix}{d}_{1 x} & {d}_{2 x} & {d}_{3 x} \\ {d}_{1 y} & {d}_{2 y} & {d}_{3 y} \\ {d}_{1 z} & {d}_{2 z} & {d}_{3 z}\end{matrix} \} = \{ \begin{matrix}{C}_{11} & {C}_{12} & {C}_{13} \\ {C}_{21} & {C}_{22} & {C}_{23} \\ {C}_{31} & {C}_{32} & {C}_{33}\end{matrix} \} \cdot \{ \begin{matrix}{F}_{1 x} & {F}_{2 x} & {F}_{3 x} \\ {F}_{1 y} & {F}_{2 y} & {F}_{3 y} \\ {F}_{1 z} & {F}_{2 z} & {F}_{3 z}\end{matrix} \} \qquad Equation 6$$

Accordingly, the compliance matrix can be calculated by:

$$\{ \begin{matrix}{C}_{11} & {C}_{12} & {C}_{13} \\ {C}_{21} & {C}_{22} & {C}_{23} \\ {C}_{31} & {C}_{32} & {C}_{33}\end{matrix} \} = \{ \begin{matrix}{d}_{1 x} & {d}_{2 x} & {d}_{3 x} \\ {d}_{1 y} & {d}_{2 y} & {d}_{3 y} \\ {d}_{1 z} & {d}_{2 z} & {d}_{3 z}\end{matrix} \} \cdot {\{ \begin{matrix}{F}_{1 x} & {F}_{2 x} & {F}_{3 x} \\ {F}_{1 y} & {F}_{2 y} & {F}_{3 y} \\ {F}_{1 z} & {F}_{2 z} & {F}_{3 z}\end{matrix} \}}^{- 1} \qquad Equation 7$$

Once the compliance matrix \tilde{C} is obtained, the stiffness matrix \tilde{K}, the inversion of \tilde{C}, may be calculated. In various embodiments, the compliance matrix may be stored within a computer-readable data storage unit.

The calculation is much simpler where the 3 calibration forces are exerted along the 3 principal axes—resulting in a simple form force matrix:

$$\{ \begin{matrix}{F}_{1 x} & {F}_{2 x} & {F}_{3 x} \\ {F}_{1 y} & {F}_{2 y} & {F}_{3 y} \\ {F}_{1 z} & {F}_{2 z} & {F}_{3 z}\end{matrix} \} = \{ \begin{matrix}{F}_{1 x} & 0 & 0 \\ 0 & {F}_{2 y} & 0 \\ 0 & 0 & {F}_{3 z}\end{matrix} \} \qquad Equation 8$$

A force at any orientation can be calculated using Eq. 4 with a stiffness matrix \tilde{K} obtained from the calibration step. The displacements in the equation are measured values at known forces.

It is important to note that the above calibration equations for a three point measuring system are only accurate when forces are exerted on the distal tip of the catheter through the same point. That is, the forces during calibration must go through the same point, and forces in the subsequent measurements must also be applied to the point where the calibration forces were applied. This is critical for accuracy. However, during in vivo use of the catheter system it is not always practical to position the end effector 88 in such a way as to apply the force to the calibration point. As a result, the force measurement of a force sensor assembly calibrated in the above manner is not very accurate.

A Force Measurement System that Compensates for a Bending Moment

During use of a force sensing catheter system in vivo, lateral contact between a distal tip (end effector) of the catheter and tissue induces a bending moment on the catheter and deformable body therein. A bending moment is absent only when force exerted on the distal tip of the catheter is exclusively axially loaded. To consider the effect of a bending moment on the force calculations discussed above, Eq. 1 may be rewritten as:

D= [symbol] ·F+ [symbol] ·M Equation 9

where [symbol] is a compliance matrix associated with force, [symbol] is a compliance matrix associated with moment, and M is the moment. In a catheter, there is a co-axial twist moment, so M is a two dimension vector with non-zero components of Mₓ and M_{y} and M_{z}=0. The twist moment components are the inputs in Eq. (9). Solving for [symbol] and [symbol] is discussed below.

In a three point force measurement system as discussed above, the system may include 3 force sensing elements, such as fiber optic force sensing elements, which measure the z-direction displacement in 3 different positions. Without considering a bending moment, the \tilde{C} and \tilde{K} matrices in Eq. 1 and Eq. 2 can be completely determined by performing calibration loadings on the distal tip of the catheter in each of three axial planes. However in Eq. 9, there are two additional terms in [symbol] , accordingly two more tests are required in order to determine [symbol] . Therefore, a total of 5 calibration tests are required to completely determine the [symbol] and [symbol] in this case. Referring to FIG. 5, these 5 loading conditions may be axial loading (F_{Z}), and lateral loading along a distal plane (F_{X}^{d} and F_{Y}^{d}) and proximal plane (F_{X}^{p} and F_{Y}^{p}), respectively. With these two additional calibration tests, both [symbol] and [symbol] may be calculated.

Similar to Eq. 3, an expanded format of Eq. 9 can be written as:

$$\{ \begin{matrix}{d}_{1} \\ {d}_{2} \\ {d}_{3}\end{matrix} \} = \{ \begin{matrix}{C}_{F 11} & {C}_{F 12} & {C}_{F 13} \\ {C}_{F 21} & {C}_{F 22} & {C}_{F 23} \\ {C}_{F 31} & {C}_{F 32} & {C}_{F 33}\end{matrix} \} \cdot \{ \begin{matrix}{F}_{x} \\ {F}_{y} \\ {F}_{z}\end{matrix} \} + \{ \begin{matrix}{C}_{M 11} & {C}_{M 12} \\ {C}_{M 21} & {C}_{M 21} \\ {C}_{M 31} & {C}_{M 31}\end{matrix} \} \cdot \{ \begin{matrix}{M}_{x} \\ {M}_{y}\end{matrix} \} \qquad Equation 10$$

When the lateral forces are applied on distal plane (F_{X}^{d} and F_{Y}^{d} in FIG. 5), the displacement measured by the 3 optical fibers are:

$$\{ \begin{matrix}{d}_{11}^{d} & {d}_{12}^{d} \\ {d}_{21}^{d} & {d}_{22}^{d} \\ {d}_{31}^{d} & {d}_{32}^{d}\end{matrix} \} = \{ \begin{matrix}{C}_{F 11} & {C}_{F 12} \\ {C}_{F 21} & {C}_{F 22} \\ {C}_{F 31} & {C}_{F 32}\end{matrix} \} \cdot \{ \begin{matrix}{F}_{x}^{d} & 0 \\ 0 & {F}_{y}^{d}\end{matrix} \} + \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}0 & {M}_{x}^{d} \\ {M}_{y}^{d} & 0\end{matrix} \} \qquad Equation 11$$

When the third row of [symbol] (axial component) is removed because the axial force component is equal to zero. The forces applied along the proximal plane are:

$$\{ \begin{matrix}{d}_{11}^{p} & {d}_{12}^{p} \\ {d}_{21}^{p} & {d}_{22}^{p} \\ {d}_{31}^{p} & {d}_{32}^{p}\end{matrix} \} = \{ \begin{matrix}{C}_{F 11} & {C}_{F 12} \\ {C}_{F 21} & {C}_{F 22} \\ {C}_{F 31} & {C}_{F 32}\end{matrix} \} \cdot \{ \begin{matrix}{F}_{x}^{p} & 0 \\ 0 & {F}_{y}^{p}\end{matrix} \} + \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}0 & {M}_{x}^{p} \\ {M}_{y}^{p} & 0\end{matrix} \} \qquad Equation 12$$

Note that in the above two equations (Eqs. 11 and 12), force applied along an x direction causes a bending moment along a y direction, and a force applied along a y direction causes a bending moment along an x direction. The result of subtracting Eq. 11 from Eq. 12 is:

$$\{ \begin{matrix}{d}_{11}^{p} - {d}_{11}^{d} & {d}_{12}^{p} - {d}_{12}^{d} \\ {d}_{21}^{p} - {d}_{21}^{d} & {d}_{22}^{p} - {d}_{22}^{d} \\ {d}_{31}^{p} - {d}_{31}^{d} & {d}_{32}^{p} - {d}_{32}^{d}\end{matrix} \} = \{ \begin{matrix}{C}_{F 11} & {C}_{F 12} \\ {C}_{F 21} & {C}_{F 22} \\ {C}_{F 31} & {C}_{F 32}\end{matrix} \} \cdot \{ \begin{matrix}{F}_{x}^{p} - {F}_{x}^{d} & 0 \\ 0 & {F}_{y}^{p} - {F}_{y}^{d}\end{matrix} \} + \{ \begin{matrix}{C}_{M 11} & {C}_{M 12} \\ {C}_{M 21} & {C}_{M22} \\ {C}_{M 31} & {C}_{M 32}\end{matrix} \} \cdot \{ \begin{matrix}0 & {M}_{x}^{p} - {M}_{x}^{d} \\ {M}_{y}^{p} - {M}_{y}^{d} & 0\end{matrix} \} \qquad Equation 13$$

When the applied forces are the same along both a proximal plane and a distal plane (i.e. Fₓ^{d}=Fₓ^{p}=F_{y}^{d}=F_{y}^{p}), Eq. 13 can be simplified as:

$$\{ \begin{matrix}{d}_{11}^{p} - {d}_{11}^{d} & {d}_{12}^{p} - {d}_{12}^{d} \\ {d}_{21}^{p} - {d}_{21}^{d} & {d}_{22}^{p} - {d}_{22}^{d} \\ {d}_{31}^{p} - {d}_{31}^{d} & {d}_{32}^{p} - {d}_{32}^{d}\end{matrix} \} = \{ \begin{matrix}{C}_{M 11} & {C}_{M 12} \\ {C}_{M 21} & {C}_{M22} \\ {C}_{M 31} & {C}_{M 32}\end{matrix} \} \cdot \{ \begin{matrix}0 & {M}_{x}^{p} - {M}_{x}^{d} \\ {M}_{y}^{p} - {M}_{y}^{d} & 0\end{matrix} \} \qquad Equation 14$$

Accordingly, the [symbol] matrix is:

$$\{ \begin{matrix}{C}_{M 11} & {C}_{M 12} \\ {C}_{M 21} & {C}_{M22} \\ {C}_{M 31} & {C}_{M 32}\end{matrix} \} = {\{ \begin{matrix}0 & {M}_{x}^{p} - {M}_{x}^{d} \\ {M}_{y}^{p} - {M}_{y}^{d} & 0\end{matrix} \}}^{- 1} \cdot \{ \begin{matrix}{d}_{11}^{p} - {d}_{11}^{d} & {d}_{12}^{p} - {d}_{12}^{d} \\ {d}_{21}^{p} - {d}_{21}^{d} & {d}_{22}^{p} - {d}_{22}^{d} \\ {d}_{31}^{p} - {d}_{31}^{d} & {d}_{32}^{p} - {d}_{32}^{d}\end{matrix} \} \qquad Equation 15$$

and the [symbol] matrix is:

$$\{ \begin{matrix}{C}_{F 11} & {C}_{F 12} \\ {C}_{F 21} & {C}_{F 22} \\ {C}_{F 31} & {C}_{F 32}\end{matrix} \} = ( \{ \begin{matrix}{d}_{11}^{p} & {d}_{12}^{p} \\ {d}_{21}^{p} & {d}_{22}^{p} \\ {d}_{31}^{p} & {d}_{32}^{p}\end{matrix} \} - \{ \begin{matrix}{C}_{M 11} & {C}_{M 12} \\ {C}_{M 21} & {C}_{M22} \\ {C}_{M 31} & {C}_{M 32}\end{matrix} \} \cdot \{ \begin{matrix}0 & {M}_{x}^{p} \\ {M}_{y}^{p} & 0\end{matrix} \} ) \cdot {\{ \begin{matrix}{F}_{x}^{p} & 0 \\ 0 & {F}_{y}^{p}\end{matrix} \}}^{- 1} \qquad Equation 16$$

By adding the axial component back in to the equation, any force can be calculated using:

$$\{ \begin{matrix}{F}_{x} \\ {F}_{y} \\ {F}_{z}\end{matrix} \} = ( \{ \begin{matrix}{d}_{1} \\ {d}_{2} \\ {d}_{3}\end{matrix} \} - \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}{M}_{x} \\ {M}_{y}\end{matrix} \} ) \cdot {\{ \begin{matrix}{C}_{F 11} & {C}_{F 12} & {C}_{F 13} \\ {C}_{F 21} & {C}_{F 22} & {C}_{F 23} \\ {C}_{F 31} & {C}_{F 32} & {C}_{F 33}\end{matrix} \}}^{- 1} \qquad Equation 17$$

Eq. 17 is the force calculation formula in a 3-point measurement system. In this equation [symbol] is known from Eq. 15, [symbol] is calculated from Eq. 16, (d₁, d₂, d₃) are the displacements measured by 3 optical fibers. (Mₓ, M_{y}) are new in this equation and should be known in order to calculate the forces.

It is to be understood that the three point measurement system and the calibration matrices, disclosed herein, may be readily adapted for force measurement systems with one or two sensor configurations. With a two point measurement system, for example, the calibration matrix may still provide improved force sensing accuracy (with a force vector determination limited to a single plane), and account for a moment force in at least one plane of the catheter. A calibration matrix adapted to facilitate a single point measurement system may not be capable of detecting a vector of a force exerted on a distal tip of a catheter, or a moment on the distal tip associated with the exerted forced; however, the accuracy of the single point measurement system's force magnitude determination may still be improved.

### Specific/Experimental Results—Three Point Measurement System

Finite Element Analysis (FEA) was used to validate the accuracy of Equations 4 and 17.

a. Model Assembly and Loading Conditions

The FEA model includes a deformable body and an end effector, see FIG. 5. Five simulations were run to serve as calibration cases. Referring to FIG. 5, these 5 loading conditions were axial loading (F_{Z}), and lateral loading along a distal plane (F_{X}^{d} and F_{Y}^{d}) and proximal plane (F_{X}^{p} and F_{Y}^{p}), respectively. The distal (“D”) and proximal (“P”) planes, as shown in FIG. 5, are approximately 1 millimeter apart. The force amplitude for each loading simulation was 50 grams. The displacements and moments of these 5 cases are listed in Table 1, below. The displacement is in nano-meters, and the moment is in Newton-meters.

TABLE 1
Force and moment measurements

| | Distal | Distal | | Proximal | Proximal |
|---|---|---|---|---|---|
| | 0-90 | 0-0 | Axial | 0-90 | 0-0 |
| Fiber 1 | 844.2111 | 1162.4884 | −130.6550 | 550.1232 | 758.7246 |
| Fiber 2 | 1274.0736 | 810.9280 | −136.9272 | 833.6735 | 525.7872 |
| Fiber 3 | −1021.2707 | −1403.3710 | −149.0759 | −709.0881 | −967.2365 |
| Mx | 0.000 | 2.476 | 0.000 | 0.000 | 1.961 |
| My | 2.476 | 0.000 | 0.000 | 1.961 | 0.000 |

Lateral forces, along a third plane between proximal and distal planes, D and P, respectively in FIG. 5, were also added to the FEA simulation and the results used to check the accuracy of Eq. 4 and Eq. 17.

b. Force Results in Accordance with Equation 4

Using axial loading and two distal plane lateral loadings in calibration, the compliance matrix \tilde{C} is:

$$\tilde{C} = \{ \begin{matrix}- 16.8842 & - 23.2498 & 2.6131 \\ - 25.4815 & - 16.2186 & 2.7385 \\ 20.4254 & 28.0674 & 2.9815\end{matrix} \} \qquad Equation 18$$

Accordingly, the stiffness matrix is:

$$\tilde{K} = {\tilde{C}}^{- 1} = \{ \begin{matrix}0.06402 & - 0.074294 & 0.01089 \\ - 0.06744 & 0.05303 & 0.01040 \\ 0.19630 & 0.00051 & 0.16289\end{matrix} \} \qquad Equation 19$$

The forces calculated (in grams) by Eq. 4 are listed in Table 2, below.

TABLE 2
Calculated force measurements using a three point calibration method

| | Distal | Distal | | Mid | Mid | Proximal | Proximal |
|---|---|---|---|---|---|---|---|
| | 0-90 | 0-0 | Axial | 0-90 | 0-0 | 0-90 | 0-0 |
| Fx | −50.00 | 0.00 | 0.00 | −42.05 | −0.14 | −33.31 | −0.30 |
| Fy | 0.00 | −50.00 | 0.00 | −0.13 | −42.07 | −0.27 | −33.35 |
| Fz | 0.00 | 0.00 | −50.00 | −3.38 | −3.98 | −7.10 | −8.36 |

The forces in Table 2 are simulated, measured forces. Each of the applied forces are 50 grams. However, only the axial force and the forces exerted on the distal plane, D, exhibit good accuracy. All the forces applied on the other two planes, proximal plane P and a mid-point plane, exhibit undesirably large errors. The results suggest that the further away from the calibration plane that a force is exerted, the higher the force measurement error. The reason for this discrepancy is that the forces at the mid-point plane and proximal plane P are not exerted through the same point as the forces at the distal plane D, and therefore these forces exhibit bending moments at the distal plane D.

Similar results can be seen using the proximal plane P as the calibration plane, or the mid-point plane as the calibration plane. Accordingly, in the three point calibration method, the force measurement is accurate only if the force is exerted at the same plane that the calibration is conducted.

### Specific/Experimental Results—Five Point Measurement System

Five calibration measurements are required when using Eq. 17 to calculate forces. In this FEA-based study, the axial loading, two lateral loadings along a distal plane, and two lateral loadings along a proximal plane are used for calibration. Using the data in Table 1, the [symbol] is

$$\begin{matrix}= \{ \begin{matrix}571.0445 & 784.0074 \\ 855.1458 & 553.6715 \\ - 606.180 & - 846.863\end{matrix} \} \\ and & Equation 20 \\ = \{ \begin{matrix}0.10059 & - 0.11652 & 0.018865 \\ - 0.1079 & 0.083202 & 0.018135 \\ 0.17825 & - 0.01216 & 0.190344\end{matrix} \} & Equation 21\end{matrix}$$

The forces (in grams) calculated by Eq. 17 are listed in Table 3, below.

TABLE 3
Force measurement (calculated) using five-point calibration method

| | Proximal | Proximal | | Mid | Mid | Distal | Distal |
|---|---|---|---|---|---|---|---|
| | 0-90 | 0-0 | Axial | 0-90 | 0-0 | 0-90 | 0-0 |
| Fx | 50.00 | 0.00 | 0.00 | 50.01 | 0.00 | 50.00 | 0.00 |
| Fy | 0.00 | 50.00 | 0.00 | 0.00 | 50.01 | 0.00 | 50.00 |
| Fz | 0.00 | 0.00 | −50.00 | 0.01 | 0.01 | 0.00 | 0.00 |

By considering bending moments when calculating an estimated force measurement, in accordance with Eq. 17, the calculated force measurements show vastly improved accuracy. Importantly, Equation 17 considers both the bending moments in the calibration planes and the offset planes. These results demonstrate that accounting for bending, when determining a force exerted on a catheter tip, is highly desirable.

The results of Table 3 evidence that the force measurement can be greatly improved by considering bending. However, such an equation requires more information, including: a 5 point calibration test, and/or that the bending moments are known value(s) in a 3-point measurement system.

When adding bending into the calibration equation, there are 3 force components and two bending components, a total of 5 variables. All 5 variables may be determined if there are 5 calibration measurements of the deformable body. The equation for such a calibration is:

$$\{ \begin{matrix}{d}_{1} \\ {d}_{2} \\ {d}_{3} \\ {d}_{4} \\ {d}_{5}\end{matrix} \} = \{ \begin{matrix}{C}_{11} & {C}_{12} & {C}_{13} & {C}_{14} & {C}_{15} \\ {C}_{21} & {C}_{22} & {C}_{23} & {C}_{24} & {C}_{25} \\ {C}_{31} & {C}_{32} & {C}_{33} & {C}_{34} & {C}_{35} \\ {C}_{41} & {C}_{42} & {C}_{43} & {C}_{44} & {C}_{45} \\ {C}_{51} & {C}_{52} & {C}_{53} & {C}_{54} & {C}_{55}\end{matrix} \} \cdot \{ \begin{matrix}{F}_{x} \\ {F}_{y} \\ {F}_{z} \\ {M}_{x} \\ {M}_{y}\end{matrix} \} \qquad Equation 22$$

The (d₁, d₂, d₃, d₄, d₅) are the measured displacements of the 5 force sensing elements, the 5×5 \tilde{C} matrix is a new compliance matrix, and the force vector includes forces and moments. Equation 22 also requires 5 calibration measurements. The force and moment are input during the calibration test. Therefore the \tilde{C} matrix can be determined by calibration.

Once the \tilde{C} matrix is obtained, the forces and moments can be easily calculated as:

$$\{ \begin{matrix}{F}_{x} \\ {F}_{y} \\ {F}_{z} \\ {M}_{x} \\ {M}_{y}\end{matrix} \} = {\{ \begin{matrix}{C}_{11} & {C}_{12} & {C}_{13} & {C}_{14} & {C}_{15} \\ {C}_{21} & {C}_{22} & {C}_{23} & {C}_{24} & {C}_{25} \\ {C}_{31} & {C}_{32} & {C}_{33} & {C}_{34} & {C}_{35} \\ {C}_{41} & {C}_{42} & {C}_{43} & {C}_{44} & {C}_{45} \\ {C}_{51} & {C}_{52} & {C}_{53} & {C}_{54} & {C}_{55}\end{matrix} \}}^{- 1} \cdot \{ \begin{matrix}{d}_{1} \\ {d}_{2} \\ {d}_{3} \\ {d}_{4} \\ {d}_{5}\end{matrix} \} \qquad Equation 23$$

FEA may be used to validate Equation 23. The FEA analysis utilizes a model with a deformable body with integrated fiber optic force sensor assembly including five fiber optic force sensing elements distributed circumferentially about a longitudinal axis of a catheter shaft.

The axial loading F_{C1}, two lateral loadings at the proximal plane P (F_{C4-5}) and two lateral loadings from the distal plane D (F_{C2-3}) are used for calibration testing. The 5 readings from each of the sensing element in response to the loading conditions are summarized in Table 4, the input force (grams) and moments (Newton-meter) are summarized in Table 5, and the calculated forces (grams) and moments (Newton-meter) calculated by Eq. 23 are listed in Table 6.

TABLE 4
Sensing element readings during calibration test

| | Distal | Distal | | Proximal | Proximal |
|---|---|---|---|---|---|
| | 0-90 | 0-0 | Axial | 0-90 | 0-0 |
| Fiber 1 | 844.2111 | 1162.4884 | −130.6550 | 550.1232 | 758.7246 |
| Fiber 2 | 1274.0736 | 810.9280 | −136.9272 | 833.6735 | 525.7872 |
| Fiber 3 | −1021.2707 | −1403.3710 | −149.0759 | −709.0881 | −967.2365 |
| Reading 4 | 1351.9500 | 1267.6600 | −146.3590 | 886.7670 | 828.9600 |
| Reading 5 | −1529.5900 | −1506.8800 | −163.0720 | −1053.6300 | −1037.9800 |

TABLE 5
The five loading cases in calibration

| | Distal | Distal | | Proximal | Proximal |
|---|---|---|---|---|---|
| | 0-90 | 0-0 | Axial | 0-90 | 0-0 |
| Fx | 50.000 | 0.000 | 0.000 | 50.000 | 0.000 |
| Fy | 0.000 | 50.000 | 0.000 | 0.000 | 50.000 |
| Fz | 0.000 | 0.000 | 50.000 | 0.000 | 0.000 |
| Mx | 0.000 | 2.476 | 0.000 | 0.000 | 1.961 |
| My | 2.476 | 0.000 | 0.000 | 1.961 | 0.000 |

TABLE 6
Force measurement calculated using 5-point measurement calibration method

| | Distal | Distal | | Mid | Mid | Proximal | Proximal | Tilt | Tilt |
|---|---|---|---|---|---|---|---|---|---|
| | 0-90 | 0-0 | Axial | 0-90 | 0-0 | 0-90 | 0-0 | 45-90 | 45-0 |
| Fx | −50.00 | 0.00 | 0.00 | −41.11 | 0.85 | −50.00 | 0.00 | −34.14 | 1.35 |
| Fy | 0.00 | −50.00 | 0.00 | 0.80 | −49.23 | 0.00 | −50.00 | 1.23 | −34.38 |
| Fz | 0.00 | 0.00 | −50.00 | −0.79 | −0.76 | 0.00 | 0.00 | 36.51 | −36.45 |
| Mx | 0.00 | 2.48 | 0.00 | −0.01 | 2.22 | 0.00 | 1.96 | −0.02 | 1.73 |
| My | 2.48 | 0.00 | 0.00 | 2.21 | −0.02 | 1.96 | 0.00 | 1.73 | −0.02 |

The results in Table 6 demonstrate that the 5-point measurement/calibration system utilizing Equation 23 achieves accurate force measurements. Two more loading conditions with 45° tilt were simulated to further validate the method disclosed above, the results are listed in Table 6. At a 45° tilt loading orientation, the force components should be 35.35 grams.

Although several embodiments have been described above with a certain degree of particularity, those skilled in the art could make numerous alterations to the disclosed embodiments without departing from the spirit of the present disclosure. It is intended that all matter contained in the above description or shown in the accompanying drawings shall be interpreted as illustrative only and not limiting. Changes in detail or structure may be made without departing from the present teachings. The foregoing description and following claims are intended to cover all such modifications and variations.

Various embodiments are described herein of various apparatuses, systems, and methods. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and do not necessarily limit the scope of the embodiments, the scope of which is defined solely by the appended claims.

Reference throughout the specification to “various embodiments,” “some embodiments,” “one embodiment,” “an embodiment,” or the like, means that a particular feature, structure, or characteristic described in connection with the embodiment is included in at least one embodiment. Thus, appearances of the phrases “in various embodiments,” “in some embodiments,” “in one embodiment,” “in an embodiment,” or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. A force-sensing catheter system comprising:
a catheter including
a distal tip,
a deformable body coupled near the distal tip, and configured and arranged to deform in response to a force exerted on the distal tip, and
a force sensor, including three or more sensing elements, configured and arranged to detect the deformation of the deformable body and transmit a signal indicative of the deformation; and
processing circuitry configured and arranged to receive the signal from the force sensor, and to determine a magnitude of the force exerted on the catheter tip that accounts for a bending moment associated with the force exerted.

2. The force-sensing catheter system of claim 1, further including
a display communicatively coupled to the processing circuitry and configured and arranged to visually indicate to a clinician the force exerted on the distal tip of the catheter.

3. The force-sensing catheter system of claim 1, wherein the force sensor includes five or more sensing elements.

4. The force-sensing catheter system of claim 3, wherein the five or more sensing elements are circumferentially distributed about a longitudinal axis of the catheter.

5. The force-sensing catheter system of claim 1, wherein the sensing elements are fiber optic sensing elements, and the processing circuitry is further configured and arranged to determine a vector of the force exerted on the catheter tip.

6. The force-sensing catheter system of claim 3, wherein the processing circuitry is further configured and arranged to calibrate the force sensor by determining a first compliance matrix associated with force ( [symbol] ) and a second compliance matrix associated with a moment ( [symbol] ).

7. The force-sensing catheter system of claim 6, wherein determining the [symbol] and the [symbol] matrices requires at least five known applications of a force at known points along the distal tip of the catheter.

8. The force-sensing catheter system of claim 1, wherein the processing circuitry is configured and arranged to determine the magnitude of the force exerted on the catheter tip using the equation: D= [symbol] ·F+ [symbol] ·M, where D is the displacement vector, F is the force vector, [symbol] is a first compliance matrix associated with the force vector F, and [symbol] is a second compliance matrix associated with a moment M.

9. The force-sensing catheter system of claim 1, wherein the processing circuitry is further configured and arranged to determine the magnitude and a vector of the force exerted on the catheter tip using the equation:
$$\{ \begin{matrix}{F}_{x} \\ {F}_{y} \\ {F}_{z}\end{matrix} \} = ( \{ \begin{matrix}{d}_{1} \\ {d}_{2} \\ {d}_{3}\end{matrix} \} - \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}{M}_{x} \\ {M}_{y}\end{matrix} \} ) \cdot {\{ \begin{matrix}{C}_{F 11} & {C}_{F 12} & {C}_{F 13} \\ {C}_{F 21} & {C}_{F 22} & {C}_{F 23} \\ {C}_{F 31} & {C}_{F 32} & {C}_{ F 33}\end{matrix} \}}^{- 1} ,$$
where the force sensor includes three sensing elements, d₁, d₂, d₃ are the displacements measured by the three sensing elements, and the moments Mₓ, M_{y} are known.

10. The force-sensing catheter system of claim 9, wherein the [symbol] matrix is calculated using the equation:
$$\{ \begin{matrix}{C}_{M 11} & {C}_{M 12} \\ {C}_{M 21} & {C}_{M 22} \\ {C}_{M 31} & {C}_{M 32}\end{matrix} \} = {\{ \begin{matrix}0 & {M}_{x}^{p} - {M}_{x}^{d} \\ {M}_{y}^{p} - {M}_{y}^{d} & 0\end{matrix} \}}^{- 1} \cdot \{ \begin{matrix}{d}_{11}^{p} - {d}_{11}^{d} & {d}_{12}^{p} - {d}_{12}^{d} \\ {d}_{21}^{p} - {d}_{21}^{d} & {d}_{22}^{p} - {d}_{22}^{d} \\ {d}_{31}^{p} - {d}_{31}^{d} & {d}_{32}^{p} - {d}_{32}^{d}\end{matrix} \} .$$

11. The force-sensing catheter system of claim 10, wherein the [symbol] matrix is calculated using the equation:
$$\{ \begin{matrix}{C}_{F 11} & {C}_{F 12} \\ {C}_{F 21} & {C}_{F 22} \\ {C}_{F 31} & {C}_{F 32}\end{matrix} \} = ( \{ \begin{matrix}{d}_{11}^{p} & {d}_{12}^{p} \\ {d}_{21}^{p} & {d}_{22}^{p} \\ {d}_{31}^{p} & {d}_{32}^{p}\end{matrix} \} - \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}0 & {M}_{x}^{p} \\ {M}_{y}^{p} & 0\end{matrix} \} ) \cdot {\{ \begin{matrix}{F}_{x}^{p} & 0 \\ 0 & {F}_{y}^{p}\end{matrix} \}}^{- 1} .$$

12. The force-sensing catheter system of claim 3, wherein the processing circuitry is configured and arranged to determine a compliance matrix, 5×5 \tilde{C}, during calibration using the equation:
$$\{ \begin{matrix}{d}_{1} \\ {d}_{2} \\ {d}_{3} \\ {d}_{4} \\ {d}_{5}\end{matrix} \} = \{ \begin{matrix}{C}_{11} & {C}_{12} & {C}_{13} & {C}_{14} & {C}_{15} \\ {C}_{21} & {C}_{22} & {C}_{23} & {C}_{24} & {C}_{25} \\ {C}_{31} & {C}_{32} & {C}_{33} & {C}_{34} & {C}_{35} \\ {C}_{41} & {C}_{42} & {C}_{43} & {C}_{44} & {C}_{45} \\ {C}_{51} & {C}_{52} & {C}_{53} & {C}_{54} & {C}_{55}\end{matrix} \} \cdot \{ \begin{matrix}{F}_{x} \\ {F}_{y} \\ {F}_{z} \\ {M}_{x} \\ {M}_{y}\end{matrix} \} ,$$
where d₁, d₂, d₃, d₄, d₅ are the measured displacements of the five sensing elements during the calibration, and the calibration forces and moments applied to the force sensor are known.

13. The force-sensing catheter system of claim 12, wherein the calibration requires at least five known applications of a force at known points along the distal tip of the catheter.

14. The force-sensing catheter system of claim 3, wherein the processing circuitry is configured and arranged to determine the magnitude and vector of the force and moments exerted on the catheter tip using the equation:
$$\{ \begin{matrix}{F}_{x} \\ {F}_{y} \\ {F}_{z} \\ {M}_{x} \\ {M}_{y}\end{matrix} \} = {\{ \begin{matrix}{C}_{11} & {C}_{12} & {C}_{13} & {C}_{14} & {C}_{15} \\ {C}_{21} & {C}_{22} & {C}_{23} & {C}_{24} & {C}_{25} \\ {C}_{31} & {C}_{32} & {C}_{33} & {C}_{34} & {C}_{35} \\ {C}_{41} & {C}_{42} & {C}_{43} & {C}_{44} & {C}_{45} \\ {C}_{51} & {C}_{52} & {C}_{53} & {C}_{54} & {C}_{55}\end{matrix} \}}^{- 1} \cdot \{ \begin{matrix}{d}_{1} \\ {d}_{2} \\ {d}_{3} \\ {d}_{4} \\ {d}_{5}\end{matrix} \} ,$$
where the \tilde{C} matrix is a known compliance matrix, and d₁, d₂, d₃, d₄, d₅ are the measured displacements of the five sensing elements.

15. The force-sensing catheter system of claim 14, wherein the C matrix is determined by calibration of the force sensor, the calibration including an axial loading of the distal tip of the catheter, two lateral loadings at a proximal plane of the distal tip, and two lateral loadings at a distal plane of the distal tip.

16. A calibration method for a force-sensing catheter system including:
successively applying forces at designated points along a distal tip of a catheter;
based on a response of a force sensor to the force applications, determine a first compliance matrix.

17. The calibration method of claim 16, further including determining a second compliance matrix, [symbol] , associated with a moment, based on the force sensors response to the force applications; and
wherein the force sensor includes three sensing elements, and the first compliance matrix, [symbol] , is associated with a force.

18. The calibration method of claim 17, wherein [symbol] is calculated using the equation:
$$\{ \begin{matrix}{C}_{M 11} & {C}_{M 12} \\ {C}_{M 21} & {C}_{M 22} \\ {C}_{M 31} & {C}_{M 32}\end{matrix} \} = {\{ \begin{matrix}0 & {M}_{x}^{p} - {M}_{x}^{d} \\ {M}_{y}^{p} - {M}_{y}^{d} & 0\end{matrix} \}}^{- 1} \cdot \{ \begin{matrix}{d}_{11}^{p} - {d}_{11}^{d} & {d}_{12}^{p} - {d}_{12}^{d} \\ {d}_{21}^{p} - {d}_{21}^{d} & {d}_{22}^{p} - {d}_{22}^{d} \\ {d}_{31}^{p} - {d}_{31}^{d} & {d}_{32}^{p} - {d}_{32}^{d}\end{matrix} \} .$$

19. The calibration method of claim 17, wherein [symbol] is calculated using the equation:
$$\{ \begin{matrix}{C}_{F 11} & {C}_{F 12} \\ {C}_{F 21} & {C}_{F 22} \\ {C}_{F 31} & {C}_{F 32}\end{matrix} \} = ( \{ \begin{matrix}{d}_{11}^{p} & {d}_{12}^{p} \\ {d}_{21}^{p} & {d}_{22}^{p} \\ {d}_{31}^{p} & {d}_{32}^{p}\end{matrix} \} - \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}0 & {M}_{x}^{p} \\ {M}_{y}^{p} & 0\end{matrix} \} ) \cdot {\{ \begin{matrix}{F}_{x}^{p} & 0 \\ 0 & {F}_{y}^{p}\end{matrix} \}}^{- 1} .$$

20. The calibration method of claim 16, wherein successively applying forces at designated points along a distal tip of a catheter includes applying forces along a longitudinal axis of the distal tip, laterally along a proximal plane of the distal tip, and laterally along a distal plane of the distal tip.

21. The calibration method of claim 16, wherein the force sensor includes five or more sensing elements; and wherein the compliance matrix is calculated using the equation:
$$\{ \begin{matrix}{d}_{1} \\ {d}_{2} \\ {d}_{3} \\ {d}_{4} \\ {d}_{5}\end{matrix} \} = \{ \begin{matrix}{C}_{11} & {C}_{12} & {C}_{13} & {C}_{14} & {C}_{15} \\ {C}_{21} & {C}_{22} & {C}_{23} & {C}_{24} & {C}_{25} \\ {C}_{31} & {C}_{32} & {C}_{33} & {C}_{34} & {C}_{35} \\ {C}_{41} & {C}_{42} & {C}_{43} & {C}_{44} & {C}_{45} \\ {C}_{51} & {C}_{52} & {C}_{53} & {C}_{54} & {C}_{55}\end{matrix} \} \cdot \{ \begin{matrix}{F}_{x} \\ {F}_{y} \\ {F}_{z} \\ {M}_{x} \\ {M}_{y}\end{matrix} \} ,$$
where d₁, d₂, d₃, d₄, d₅ are the measured displacements at the five sensing elements during the calibration, and the calibration forces and associated moments applied to the force sensor are known.

22. A method for detecting a force and moment exerted on a distal tip of a force-sensing catheter system including:
receiving three or more signals indicative of the displacement measured on the distal tip of the force-sensing catheter; and
applying a compliance matrix to the measured displacement to determine the force and moment exerted on the distal tip.

23. The method for detecting a force exerted on a distal tip of a force-sensing catheter system of claim 22, wherein the step of receiving three or more signals indicative of the displacement measured on the distal tip of the force-sensing catheter includes receiving five signals, and the step of applying a compliance matrix to the measured displacements to determine the force and moment exerted on the distal tip uses the equation:
$$\{ \begin{matrix}{F}_{x} \\ {F}_{y} \\ {F}_{z} \\ {M}_{x} \\ {M}_{y}\end{matrix} \} = {\{ \begin{matrix}{C}_{11} & {C}_{12} & {C}_{13} & {C}_{14} & {C}_{15} \\ {C}_{21} & {C}_{22} & {C}_{23} & {C}_{24} & {C}_{25} \\ {C}_{31} & {C}_{32} & {C}_{33} & {C}_{34} & {C}_{35} \\ {C}_{41} & {C}_{42} & {C}_{43} & {C}_{44} & {C}_{45} \\ {C}_{51} & {C}_{52} & {C}_{53} & {C}_{54} & {C}_{55}\end{matrix} \}}^{- 1} \cdot \{ \begin{matrix}{d}_{1} \\ {d}_{2} \\ {d}_{3} \\ {d}_{4} \\ {d}_{5}\end{matrix} \} ,$$
where the \tilde{C} matrix is the compliance matrix.

24. The method for detecting a force exerted on a distal tip of a force-sensing catheter system of claim 22, further including visually indicating to a clinician the force exerted on the catheter tip.

25. The method for detecting a force exerted on a distal tip of a force-sensing catheter system of claim 22, wherein the signals are pulses of light transmitted through one or more optical fibers.

26. The method for detecting a force exerted on a distal tip of a force-sensing catheter system of claim 22, wherein the step of applying the compliance matrix, [symbol] , also includes applying a second compliance matrix using the equation:
$$\{ \begin{matrix}{F}_{x} \\ {F}_{y} \\ {F}_{z}\end{matrix} \} = ( \{ \begin{matrix}{d}_{1} \\ {d}_{2} \\ {d}_{3}\end{matrix} \} - \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}{M}_{x} \\ {M}_{y}\end{matrix} \} ) \cdot {\{ \begin{matrix}{C}_{F 11} & {C}_{F 12} & {C}_{F 13} \\ {C}_{F 21} & {C}_{F 22} & {C}_{F 23} \\ {C}_{F 31} & {C}_{F 32} & {C}_{ F 33}\end{matrix} \}}^{- 1} ,$$
where the force sensor includes three force sensing elements, d₁, d₂, d₃ are the displacements measured by the three force sensing elements, and the moments Mₓ, M_{y} are known.

27. The method for detecting a force exerted on a distal tip of a force-sensing catheter system of claim 26, wherein the compliance matrix, [symbol] , is associated with a moment exerted on the distal tip, and calculated using the equation:
$$\{ \begin{matrix}{C}_{M 11} & {C}_{M 12} \\ {C}_{M 21} & {C}_{M 22} \\ {C}_{M 31} & {C}_{M 32}\end{matrix} \} = {\{ \begin{matrix}0 & {M}_{x}^{p} - {M}_{x}^{d} \\ {M}_{y}^{p} - {M}_{y}^{d} & 0\end{matrix} \}}^{- 1} \cdot \{ \begin{matrix}{d}_{11}^{p} - {d}_{11}^{d} & {d}_{12}^{p} - {d}_{12}^{d} \\ {d}_{21}^{p} - {d}_{21}^{d} & {d}_{22}^{p} - {d}_{22}^{d} \\ {d}_{31}^{p} - {d}_{31}^{d} & {d}_{32}^{p} - {d}_{32}^{d}\end{matrix} \} .$$

28. The method for detecting a force exerted on a distal tip of a force-sensing catheter system of claim 26, wherein the compliance matrix, [symbol] , is associated with the force exerted on the distal tip, and the force is calculated using the equation:
$$\{ \begin{matrix}{C}_{F 11} & {C}_{F 12} \\ {C}_{F 21} & {C}_{F 22} \\ {C}_{F 31} & {C}_{F 32}\end{matrix} \} = ( \{ \begin{matrix}{d}_{11}^{p} & {d}_{12}^{p} \\ {d}_{21}^{p} & {d}_{22}^{p} \\ {d}_{31}^{p} & {d}_{32}^{p}\end{matrix} \} - \{ \begin{matrix}{C}_{M 11} & {C}_{M 12} \\ {C}_{M 21} & {C}_{M 22} \\ {C}_{M 31} & {C}_{M 32}\end{matrix} \} \cdot \{ \begin{matrix}0 & {M}_{x}^{p} \\ {M}_{y}^{p} & 0\end{matrix} \} ) \cdot {\{ \begin{matrix}{F}_{x}^{p} & 0 \\ 0 & {F}_{y}^{p}\end{matrix} \}}^{- 1} .$$

29. The calibration method of claim 16, wherein the compliance matrix is stored within a computer-readable data storage unit.
